(19) United States
(12) Patent Application Publication
(10) Pub. No.: US 2008/0221431 A1
(43) Pub. Date: Sep. 11, 2008

(54) METHOD AND APPARATUS FOR TRANSFORMING THE COORDINATE SYSTEM OF MRI-GUIDED MEDICAL EQUIPMENT TO THE COORDINATE SYSTEM OF AN MRI SYSTEM

(76) Inventors: Florian Steinmeyer, Herzogenaurach (DE); Jian Min Wang, Shenzhen (CN); Oiang Zhang, Lisle, IL (US)

Correspondence Address: SCHIFF HARDIN, LLP;PATENT DEPARTMENT, 6600 SEARS TOWER, CHICAGO, IL, 60606-6473, US

(21) Appl. No.: 11/775,299

(22) Filed: Jul. 10, 2007

(30) Foreign Application Priority Data
Jul. 10, 2006 (CN) 200610089662.6

(51) Int. Cl. A61B 5/055

(52) U.S. Cl. 600/414; 382/131

(57) ABSTRACT

In a method and an apparatus for transforming the coordinate system of MRI-guided medical equipment to the coordinate system of an MRI system MRI imaging is used to determine the rotational and translational values of the coordinate system of MRI-guided medical equipment relative to the coordinate system of an MRI system, so as to achieve the transformation. A number of fiducial markers are arranged in the MRI-guided medical equipment, and a coil winding is arranged on each fiducial marker for generating signals to determine its position in the coordinate system of the MRI system. The coil windings are serially connected and the serially connected windings are connected to the magnetic resonance system via an interface circuit.

### BACKGROUND OF THE INVENTION

1. Field of the Invention

The present invention relates to making MRI (Magnetic Resonance Imaging)-guided medical equipment compatible with an MRI system, and particularly to an apparatus and a method for coordinate system registration in this context.

2. Description of the Prior Art

MRI-guided medical equipment, when used for treating a patient, not only can provide better imaging effects but also can control the dosage accurately.

With reference to FIG. 1 shows MRI-guided medical equipment according to the example of an MRI-guided High Intensity Focused Ultrasound (HIFU) system 10. The HIFU system 10 has a focused region located within an MRI image that matches the location in a patient that needs to be treated. In this way, a variety of MRI imaging methods, such as the proton resonance frequency (PRF) switching method, can be adopted to dynamically follow the temperature change within the focused region. For this reason, it is necessary to bring the coordinate system 12 of the HIFU system 10 into registration with the coordinate system 22 of the MRI system 20 (referred to herein as transforming the coordinate system of the MRI-guided medical equipment to the coordinate system of the MRI system.

The current method for transforming the coordinate system of MRI-guided medical equipment to the coordinate system of an MRI system is to use a mechanical positioning technique, rather than by using MRI imaging for automatically transforming the coordinate system of MRI-guided medical equipment to the coordinate system of an MRI system.

A method for projecting three fiducial markers into an MRI system so as to obtain their 3D coordinate values by calculation is described in an article “A Method for Fast 3D Tracking Using Tuned Fiducial Markers and a Limited Projection Reconstruction FISP (LPR-FISP) Sequence” published in JOURNAL OF MAGNETIC RESONANCE IMAGING 14:617-627 (2001). The fiducial markers used in the article are set up in parallel, and need to be tuned separately and then to be coupled inductively to the MRI system. Furthermore there is no discussion in the article as to how to transform the coordinate system of MRI-guided medical equipment to the coordinate system of an MRI system as claimed in the 3D coordinate values of the fiducial markers obtained in the MRI system. Therefore, how to automatically transform the coordinate system of MRI-guided medical equipment to the coordinate system of an MRI system by using the MRI imaging method has become a problem which needs an urgent solution.

### SUMMARY OF THE INVENTION

An object of the present invention is to provide an apparatus for transforming the coordinate system of MRI-guided medical equipment to the coordinate system of an MRI system and a corresponding method, in which the MRI imaging is utilized for determining the rotational and translational values of the coordinate system of the MRI-guided medical equipment relative to the coordinate system of the MRI system, so as to achieve the transformation.

The above object is achieved in accordance with the present invention proposes an apparatus for transforming the coordinate system of MRI-guided medical equipment to the coordinate system of an MRI system, having a number of fiducial markers arranged at the MRI-guided medical equipment, each fiducial marker including a coil winding arranged that generates signals indicating its position in the coordinate system of the MRI system, wherein the coil windings are serially connected, and the serially connected windings are connected to the magnetic resonance system via an interface circuit. A computer of the MRI system makes the transformation from the signals generated by the aforementioned coil windings.

The interface circuit can include a tuning circuit; and the coil windings are connected by coaxial cables. Furthermore, each of the fiducial markers contains a contrast agent. Preferably, the contrast agent is gadolinium-DTPA.

In an embodiment, the apparatus can include four fiducial markers, with one of the fiducial markers being located out of the same plane as the other three.

In an embodiment, MRI-guided medical equipment is a High Intensity Focused Ultrasound system.

The above object also is achieved in accordance with the present invention by a method for transforming the coordinate system of MRI-guided medical equipment to the coordinate system of an MRI system wherein the MRI-guided medical equipment including a number of fiducial markers; and the 3D coordinate values of the fiducial markers in the coordinate system of the MRI-guided medical equipment being known. The method includes the steps of (a) detecting a number of projections of the fiducial markers with the MRI system, (b) determining the 3D coordinate values of the fiducial markers in the coordinate system of the MRI system in the projections, (c) calculating rotational and translational values required for switching the 3D coordinate values of the fiducial markers in the coordinate system of MRI-guided medical equipment and the coordinate system of said MRI system, and (d) transforming the coordinate system of the MRI-guided medical equipment to the coordinate system of the MRI system using the calculated rotational and translational values.

The present invention utilizes MRI imaging to determine the rotational and translational values of the coordinate system of the MRI-guided medical equipment relative to the coordinate system of the MRI system by only a few projections, so as to perform the corresponding transformation. In accordance with the present invention, the coil windings arranged on the fiducial markers are serially connected to form a single coil, so that it can be turned by only one tuning circuit, making it structurally simple and easy to operate.

### BRIEF DESCRIPTION OF THE DRAWINGS

FIG. 1 illustrates an embodiment of the operation of an MRI-guided HIFU system in a corresponding MRI system.

FIG. 2 illustrates the connection of the coil windings on the fiducial markers of an apparatus for transforming the coordinate system of MRI-guided medical equipment to the coordinate system of an MRI system as claimed in the present invention.

FIG. 3 is an illustration of the relative positions of the fiducial markers in FIG. 2.

FIG. 4 is an illustration of the signals of 1D Fourier transformation of the projections of the fiducial markers in the MRI system in FIG. 2.

FIG. 5A is a flowchart of an embodiment of a method for transforming the coordinate system of RI-guided medical equipment to the coordinate system of an MRI system in accordance with the present invention.

FIG. 5B is a flowchart showing details of one of the steps in FIG. 5A.

### DETAILED DESCRIPTION OF THE EMBODIMENT

In order to transform the coordinate system of MRI-guided medical equipment to the coordinate system of an MRI system, first it is necessary to determine the rotational and translational values of the coordinate system of the MRI-guided medical equipment relative to the coordinate system of said MRI system.

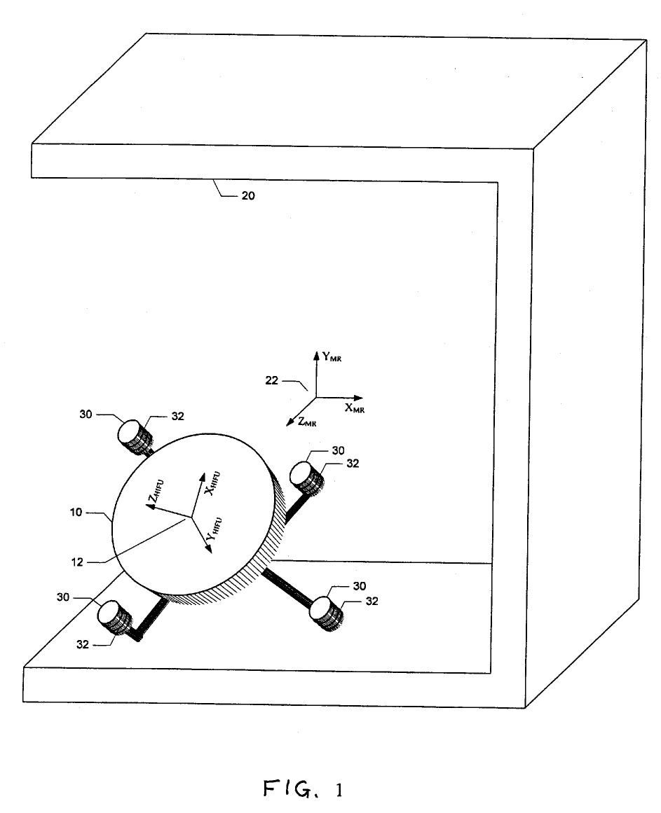

With reference to FIG. 1, still using the MRI-guided HIFU system 10 as an example for illustration, the apparatus of the present invention has a number of fiducial markers 30 arranged on the HIFU system 10. In this embodiment, the apparatus of the present invention has four fiducial markers 30, and the 3D coordinate values

$$P = [ \begin{matrix}x \\ y \\ z\end{matrix} ]$$

of the above fiducial markers 30 in the coordinate system 12 (X_{HIFU}, Y_{HIFU}, Z_{HIFU}) of the HIFU system 10 are known. As long as the 3D coordinate values

$${P}_{MR} = [ \begin{matrix}{x}_{mr} \\ {y}_{mr} \\ {z}_{mr}\end{matrix} ]$$

of the above fiducial markers 30 in the coordinate system 22 (X_{MR}, Y_{MR}, Z_{MR}) of said MRI system 20 can be determined, the rotational value

$$R = [ \begin{matrix}{R}_{1} & {R}_{2} & {R}_{3} \\ {R}_{4} & {R}_{5} & {R}_{6} \\ {R}_{7} & {R}_{8} & {R}_{9}\end{matrix} ]$$

and the translational value

$$T = [ \begin{matrix}{x}_{0} \\ {y}_{0} \\ {z}_{0}\end{matrix} ]$$

of the coordinate system 12 of the HIFU system 10 relative to the coordinate system 22 of the MRI system 20 can be calculated by the equation P=RP_{MR}+T, and thus a corresponding switching can be performed.

The present invention utilizes an MRI projection imaging method to determine the 3D coordinate values of the above fiducial markers 30 in the coordinate system 22 of the MRI system 20.

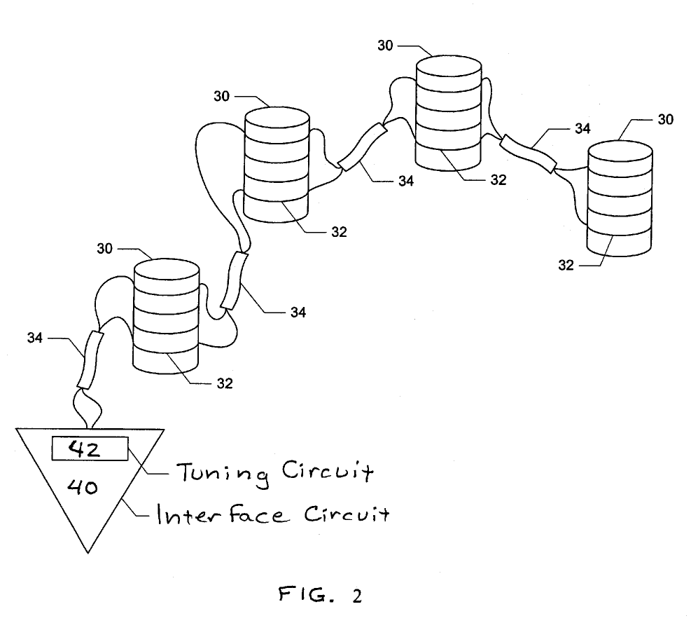

With reference to FIG. 2 at the same time, in order to obtain signals with high signal-to-noise ratio when the projections are performed, coil windings 32 are arranged respectively on the fiducial markers 30 in the apparatus of the present invention. The coil windings 32 are serially connected by coaxial cables 34 to form a single coil, which is connected to said MRI system via an interface circuit 40 so as to receive signals therefrom. The interface circuit 40 has a tuning circuit 42 for tuning the serially connected coil windings 32. Further, said fiducial markers 30 also contain a contrast agent to obtain the signals with high signal-to-noise ratio. The contrast agent can be, for example, gadolinium-DTPA (Gadolinium diethylenetriaminepentacetic acid).

Since the coil windings 32 arranged on the fiducial markers 30 are serially connected to form a single coil, only one tuning circuit 42 is needed for the tuning.

Since the rotational value

$$R = [ \begin{matrix}{R}_{1} & {R}_{2} & {R}_{3} \\ {R}_{4} & {R}_{5} & {R}_{6} \\ {R}_{7} & {R}_{8} & {R}_{9}\end{matrix} ]$$

and the translational value

$$T = [ \begin{matrix}{x}_{0} \\ {y}_{0} \\ {z}_{0}\end{matrix} ]$$

of the coordinate system 12 of the HIFU system 10 has twelve unknown values relative to the coordinate system 22 of the MRI system 20, and also since four of the fiducial markers 30 are used in this embodiment, thus at least three projections need to be performed in the MRI system 20 so as to obtain enough coordinate values of the fiducial markers 30 within the coordinate system 22 of said MRI system 20 for the equation to solve the rotational and translational values. For the convenience of the resolution and operation, it is preferable to obtain the at least three projections in respective directions that are orthogonal to one another.

Although four fiducial markers 30 within the MRI system 20 are used in this embodiment for obtaining the at least three projections in orthogonal directions, it is understood that, in other embodiments, any other number of the fiducial markers 30 can be used to perform any other number of projections in the MRI system 20, as long as enough 3D coordinate values of the fiducial markers 30 within the coordinate system 22 of the MRI system 20 can be obtained to set up said equations.

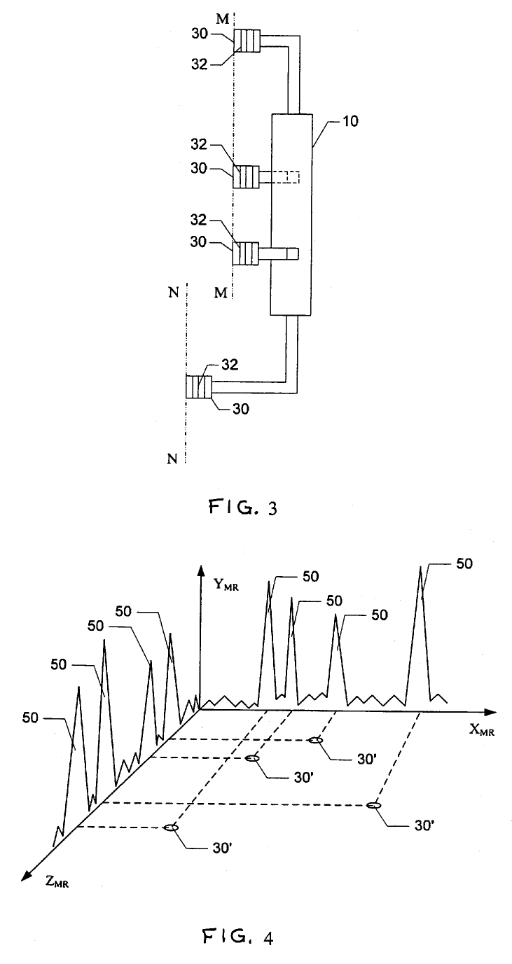

With reference to FIG. 3, in order to effectively reduce the signal superposition generated during the projections of the fiducial markers 30, among the four fiducial markers 30, one of the four fiducial markers 30 is positioned out of the same plane that is formed by the other three fiducial markers 30. For example, the lower fiducial marker 30 in FIG. 3 is located in a plane N-N, while the other three fiducial markers 30 form a plane M-M, and the planes N-N and M-M are not coplanar.

As schematically indicated in FIG. 2, the output of the interface circuit 40 is supplied to a computer 44 of the MRI system 20. The transformation from the equipment coordinate system to the MRI coordinate system is undertaken in this computer, according to the inventive method, using the signals from the fiducial markers 30.

With reference to FIG. 4, the peak positions of the 1D Fourier transformations of the fiducial markers 30 in the corresponding projection directions can be detected after the aforementioned projections have been performed according to the positions of the peaks 50 of the projections 30′ produced by the fiducial markers along the axis X_{MR} and the axis Z_{MR} shown in FIG. 4. The 3D coordinate values of the fiducial markers 30 in the coordinate system 22 of the MRI system 20 can be determined by a back-projection algorithm from these positions. Similarly, the peak positions of 1D Fourier transformations of the fiducial markers 30 in other projection directions can be also detected, and their corresponding 3D coordinate values in the coordinate system 22 of the MRI system 20 can also be determined based on the back-projection algorithm.

Since the 3D coordinate values in the coordinate system 12 of the fiducial markers 30 arranged at the HIFU system 10 are known, and the 3D coordinate values of said fiducial markers 30 in the coordinate system 22 of the MRI system 20 can be determined by using the above method, the rotational value R and the translational value T of the coordinate system 12 of the HIFU system 10 relative to the coordinate system 22 of MRI system 20 can be calculated by the equation P=RP_{MR}+T, so as to transform the coordinate system 12 of the HIFU system to the coordinate system 22 of MRI system 20 using the rotational value R and the translational value T.

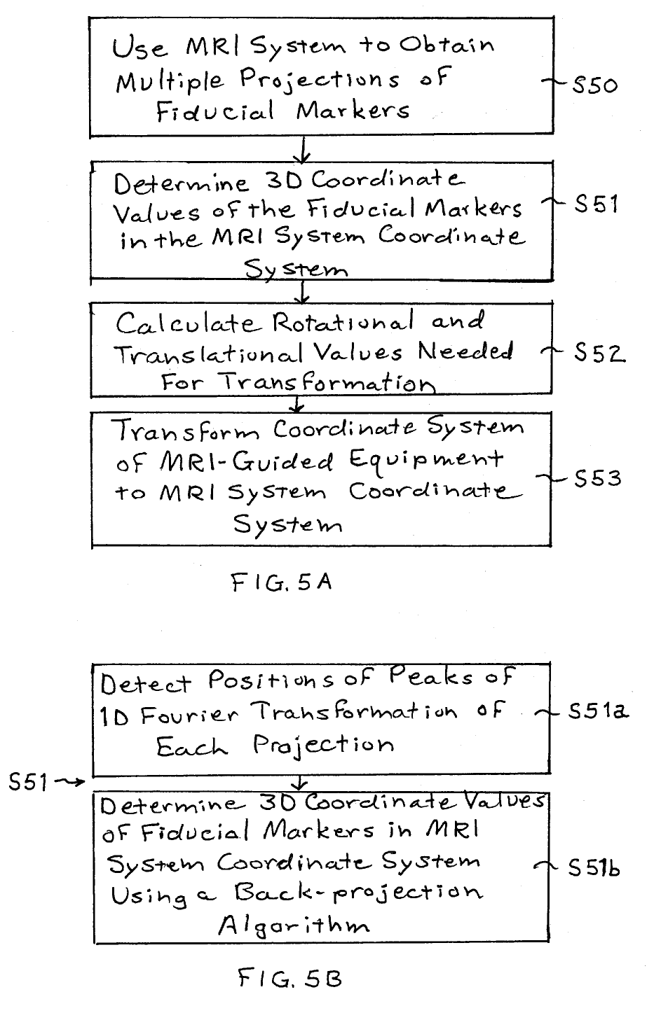

FIG. 5A is a flowchart of an embodiment of the present invention for transforming the coordinate system 12 of MRI-guided medical equipment 10 to the coordinate system 22 of an MRI system 20. The MRI-guided medical equipment 10 has a number of fiducial markers 30, with the 3D coordinate values of the fiducial markers 30 in the coordinate system 12 of the MRI-guided medical equipment 10 being known.

Step S50: using the MRI system 20 to perform a number of projections of said fiducial markers 30.

In order to obtain signals with a high signal-to-noise ratio when the projections are performed, a coil winding 32 is arranged on each of the fiducial markers 30 respectively. The coil windings 32 are serially connected by coaxial cables 34 to form a single coil which is connected to the MRI system via an interface circuit 40 so that the signals can be received. The interface circuit 40 includes a tuning circuit 42 for tuning the serially connected coil windings 32. Furthermore, the fiducial markers 30 also contain a contrast agent to generate signals with a high signal-to-noise ratio. The contrast agent can be, for example, gadolinium-DTPA. Four of the fiducial markers 30 are used in this embodiment, in which case at least three of said projections are obtained. Preferably, one of the four fiducial markers 30 is not located in the same plane as the other three, and the projections are obtained in orthogonal directions.

Step S51: The 3D coordinate values of the fiducial markers 30 in the coordinate system 22 of the MRI system 20 can be determined on the basis of the projections. As shown in FIG. 5B, Step S51 further includes:

Step S51a: detecting the positions of the peaks 50 of the 1D Fourier transformations of each projection.

Step S51b: determining the 3D coordinate values of the fiducial markers 30 in the coordinate system 22 of the MRI system 20 on the basis of a back-projection algorithm.

Step S52: The rotational and translational values required for the transformation are calculated on the basis of the 3D coordinate values of the coordinate system 12 of the fiducial markers 30 in the MRI-guided medical equipment 10 and the coordinate system 22 of the MRI system 20.

In Step 552, the rotational and translational values are calculated on the basis of the following equation:

P=RP_{MR}+T, wherein

$$P = [ \begin{matrix}x \\ y \\ z\end{matrix} ] ,$$

is the 3D coordinate values of the fiducial markers 30 in the coordinate system 12 of said MRI-guided medical equipment 10;

$${P}_{MR} = [ \begin{matrix}{x}_{mr} \\ {y}_{mr} \\ {z}_{mr}\end{matrix} ] ,$$

is the 3D coordinate values of the fiducial markers 30 in the coordinate system 22 of the MRI system 20;

$$R = [ \begin{matrix}{R}_{1} & {R}_{2} & {R}_{3} \\ {R}_{4} & {R}_{5} & {R}_{6} \\ {R}_{7} & {R}_{8} & {R}_{9}\end{matrix} ] ,$$

is the rotational value required by the switching; and

$$T = [ \begin{matrix}{x}_{0} \\ {y}_{0} \\ {z}_{0}\end{matrix} ] ,$$

is the translational value required by the switching.

Step S53: transforming the coordinate system 12 of the MRI-guided medical equipment 10 into the coordinate system 22 of the MRI system 20 as claimed in the calculated rotational value R and translational value T.

Although modifications and changes may be suggested by those skilled in the art, it is the intention of the inventor to embody within the patent warranted hereon all changes and modifications as reasonably and properly come within the scope of his contribution to the art.

We claim as our invention:

1. An apparatus for transforming a coordinate system of MRI-guided medical equipment to a coordinate system of an MRI system, comprising a plurality of fiducial markers arranged at said MRI-guided medical equipment, each said fiducial marker comprising a coil winding that generates signals that indicate its position in the coordinate system of the MRI system, said coil windings being serially connected, said MRI system comprising a computer, and the serially connected windings being are connected to said computer via an interface circuit, said computer transforming said coordinate system of said MRI-guided medical equipment to said coordinate system of said MRI system using said signals generated by the respective coil windings.

2. The apparatus as claimed in claim 1, wherein said interface circuit comprises a tuning circuit.

3. The apparatus as claimed in claim 1, wherein said coil windings are connected by coaxial cables.

4. The apparatus as claimed in claim 1, wherein each fiducial marker contains a contrast agent.

5. The apparatus as claimed in claim 4, wherein said contrast agent is gadolinium-DTPA.

6. The apparatus as claimed in claim 1, comprising four of said fiducial markers, one of the fiducial markers being arranged at said MRI-guided medical equipment in a plane outside of a plane in which the other three markers are located.

7. The apparatus as claimed in claim 1, wherein said MRI-guided medical equipment is a High Intensity Focused Ultrasound system.

8. A method for transforming the coordinate system of MRI-guided medical equipment to the coordinate system of an MRI system, said MRI-guided medical equipment comprising a plurality of fiducial markers, with the 3D coordinate values of said fiducial markers in the coordinate system of said MRI-guided medical equipment being known, said method comprising the steps of:
(a) using said MRI system to perform a plurality of projections of said fiducial markers;
(b) determining the 3D coordinate values of said fiducial markers in the coordinate system of said MRI system on the basis of said projections;
(c) calculating rotational and translational values required by the switching on the basis of the 3D coordinate values of said fiducial markers in the coordinate system of said MRI-guided medical equipment and the coordinate system of said MRI system; and
(d) automatically electronically transforming the coordinate system of said MRI-guided medical equipment to the coordinate system of said MRI system on the basis of the calculated rotational and translational values.

9. The method as claimed in claim 8, wherein step (b) further comprises:
1) detecting the peak positions of 1D Fourier transformations of each of said projections; and
2) determining the 3D coordinate values of said fiducial markers in the coordinate system of said MRI system in step (d) using a back-projection algorithm.

10. The method as claimed in claim 9, comprising elevating the rotational and translational values in step (c) according to the equation:
P=RP_{MR}+T, wherein
$$P = [ \begin{matrix}x \\ y \\ z\end{matrix} ] ,$$
is the 3D coordinate values of said fiducial markers in the coordinate system of said MRI-guided medical equipment;
$${P}_{MR} = [ \begin{matrix}{x}_{mr} \\ {y}_{mr} \\ {z}_{mr}\end{matrix} ] ,$$
is the 3D coordinate value of said fiducial markers in the coordinate system of said MRI system;
$$R = [ \begin{matrix}{R}_{1} & {R}_{2} & {R}_{3} \\ {R}_{4} & {R}_{5} & {R}_{6} \\ {R}_{7} & {R}_{8} & {R}_{9}\end{matrix} ] ,$$
the rotational value required by the switching; and
$$T = [ \begin{matrix}{x}_{0} \\ {y}_{0} \\ {z}_{0}\end{matrix} ] ,$$
is the translational value required for the transformation.

11. The method as claimed in claim 8, comprising arranging a coil winding is on each said fiducial marker, and serially connecting said coil windings.

12. The method as claimed in claim 8, comprising arranging four of said fiducial markers at said medical equipment, with one of the fiducial markers not located in the same plane as the other three.

13. The method as claimed in claim 8, comprising obtaining said projections respectively in orthogonal directions.
